(19) United States
(12) Patent Application Publication
(10) Pub. No.: US 2022/0265888 A1
(43) Pub. Date: Aug. 25, 2022

(54) AIR PURIFYING DEVICE

(71) Applicant: LENS Therapies, LLC, Charlotte, NC (US)

(72) Inventor: Loyd Frank Jarrell, Durango, CO (US)

(73) Assignee: LENS Therapies, LLC, Charlotte, NC (US)

(21) Appl. No.: 17/180,393

(22) Filed: Feb. 19, 2021

(51) Int. Cl. A61L 9/20; A61L 9/014; B01D 46/10; B01D 46/00; B01D 46/44

(52) U.S. Cl. CPC A61L 9/20; A61L 9/014; B01D 46/10; B01D 46/0023; B01D 46/0005; B01D 46/444; B01D 46/0028; B01D 46/0043; B01D 46/0049; A61L 2209/111; A61L 2209/12; A61L 2209/16; A61L 2209/14; B01D 2267/40; B01D 2273/26; B01D 2279/65; B01D 2279/50

(57) ABSTRACT

An air purifying device may comprise: a housing defining an inlet aperture and an outlet aperture; a primary filtration unit positioned within the housing, the primary filtration unit positioned proximate to the inlet aperture and configured to receive air through the inlet aperture for treatment; a secondary filtration unit positioned within the housing, the secondary filtration unit positioned proximate to the outlet aperture and configured to exhaust air through the outlet aperture; a particulate filtration unit positioned within the housing, the particulate filtration unit positioned between the primary filtration unit and the secondary filtration unit, the particulate filtration unit having an aperture that is capable of slidably receiving an ultra-low particle (ULPA) filter via an access opening; wherein an airflow pathway is provided for flow of air from the primary filtration unit to the secondary filtration unit through the particulate filtration unit.

### FIELD OF THE INVENTION

Various embodiments described herein relate generally to devices and methods for the filtration of air, and more particularly to a self-contained apparatus for air purification capable of being adapted for use in any substantially enclosed environment.

### BACKGROUND

Pollens, lung damaging dust, smoke, bacteria, viruses, dust mites, and a number of other irritants and microorganisms are commonly found in the ambient air of an enclosed area. It has long been recognized that filtration of air through some air purification means can combat some of the effects of airborne particulate contamination. There is a need for an air purifying device for removing airborne contaminate particles with an improved configuration which enhances the efficiency of removing airborne contaminate particles in an enclosed area.

### SUMMARY

The following presents a simplified summary of one or more embodiments of the present invention, in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments of the present invention in a simplified form as a prelude to the more detailed description that is presented later.

An air purifying device is provided in one embodiment, the air purifying device comprising: a housing defining an inlet aperture and an outlet aperture; a primary filtration unit positioned within the housing, the primary filtration unit positioned proximate to the inlet aperture and configured to receive air through the inlet aperture for treatment; a secondary filtration unit positioned within the housing, the secondary filtration unit positioned proximate to the outlet aperture and configured to exhaust air through the outlet aperture; a particulate filtration unit positioned within the housing, the particulate filtration unit positioned between the primary filtration unit and the secondary filtration unit, the particulate filtration unit having an aperture that is capable of slidably receiving an ultra-low particle (ULPA) filter via an access opening; wherein an airflow pathway is provided for flow of air from the primary filtration unit to the secondary filtration unit through the particulate filtration unit.

In some embodiments, the air purifying device further comprises an inlet air distributor adapted to be detachably coupled to the housing proximate to the inlet aperture, wherein the inlet air distributor is configured to direct air into the inlet aperture for treatment.

In some embodiments, the air purifying device further comprises an outlet air distributor adapted to be detachably coupled to the housing proximate to the outlet aperture, wherein the outlet air distributor is configured to direct air received from the outlet aperture.

In some embodiments, the air purifying device further comprises an access terminal positioned on the housing, the access terminal being configured for controlling operation of the primary filtration unit, the secondary filtration unit, and the particulate filtration unit.

In some embodiments, the access terminal is configured to be in network communication with at least one of a user device and/or a central server, wherein the access terminal is configured to control the operation of the primary filtration unit, the secondary filtration unit, and the particulate filtration unit through the network communication with at least the user device and/or the central server.

In some embodiments, the particulate filtration unit comprises a plurality of stabilizing elements arranged on an interior wall of the aperture, the plurality of stabilizing elements is configured to slidably receive the ultra-low particle air (ULPA) filter such that when the ultra-low particle air (ULPA) filter is inserted into the particulate filtration unit through the access opening, the plurality of stabilizing elements form a secure seal with at least a top edge and a bottom edge of the ultra-low particle air (ULPA) filter.

In some embodiments, the primary filtration unit further comprises: a high efficiency particulate air (HEPA) filter, the high efficiency particulate air (HEPA) filter positioned proximate to the inlet aperture and configured to pre-filter airborne particulate matter from the air received from the inlet aperture; an inlet airflow sensor, the airflow sensor positioned proximate to the high efficiency particulate air (HEPA) filter and configured to receive air filtered by the high efficiency particulate air (HEPA) filter; an airflow regulation device; and a plurality of ultraviolet light sources, the plurality of ultraviolet light sources is adapted to be positioned longitudinally within the primary filtration unit, wherein the airflow regulation device is configured to regulate and direct the flow of air filtered by the high efficiency particulate air (HEPA) filter towards the plurality of ultraviolet light sources.

In some embodiments, the high efficiency particulate air (HEPA) filter is configured to pre-filter airborne particulate matter of a size equal to or greater than 0.3 micron from the air received from the inlet aperture.

In some embodiments, the inlet airflow sensor is configured to measure a flow rate of air filtered by the high efficiency particulate air (HEPA) filter.

In some embodiments, the airflow regulation device is configured to regulate and direct the flow of air filtered by the high efficiency particulate air (HEPA) filter towards the plurality of ultraviolet light sources based on at least the flow rate of air measured by the inlet airflow sensor.

In some embodiments, the plurality of ultraviolet light sources is configured to generate short wave ultraviolet radiation (UV-C) to treat the air filtered by the high efficiency particulate air (HEPA) filter and displaced by the airflow regulation device.

In some embodiments, the ultra-low particle air (ULPA) filter is configured to filter airborne particulate matter from the air passed through the plurality of ultraviolet light sources in the primary filtration unit, wherein the air is moved from the primary filtration unit to the particulate filtration unit via the airflow pathway.

In some embodiments, the ultra-low particle air (ULPA) filter is configured to filter airborne particulate matter of a size equal to or greater than 0.12 micron from the air received from the primary filtration unit.

In some embodiments, the secondary filtration unit further comprises: a plurality of ultraviolet light sources, the plurality of ultraviolet light sources is adapted to be positioned longitudinally within the secondary filtration unit; an airflow regulation device; and an outlet airflow sensor, the outlet airflow sensor positioned proximate to the outlet aperture and configured to receive air from the airflow regulation device, wherein the airflow regulation device is configured to regulate and direct the flow of air from the plurality of ultraviolet light sources towards the outlet airflow sensor and the outlet aperture.

In some embodiments, the outlet airflow sensor is configured to measure a flow rate of air received from the airflow regulation device.

In some embodiments, the airflow regulation device is configured to regulate and direct the flow of air from the plurality of ultraviolet light sources towards outlet aperture based on at least the flow rate of air measured by the outlet airflow sensor.

In some embodiments, the plurality of ultraviolet light sources is configured to generate short wave ultraviolet radiation (UV-C) to treat the air received via the airflow pathway from the particulate filtration unit.

In some embodiments, the air purifying device further comprises a humidifier unit positioned within the housing, the humidifier unit configured to infuse moisture into the air prior to exhausting the air through the outlet aperture, the humidifier unit further comprising: a plurality of water filters, the plurality of water filters configured to remove waterborne contaminate matter to produce filtered water; a water basin, the water basin configured to receive filtered water from the plurality of water filters; a float valve, the float value configured to measure a water level in the water basin; and a wick cartridge, the wick cartridge configured to absorb the filtered water from the water basin, and wherein the wick cartridge is configured to infuse moisture into the air when the air is passed through the wick cartridge.

In some embodiments, the housing is adapted to be mounted in line with or intermediate of a plurality of framing members of a wall in a concealed configuration.

In some embodiments, the housing is adapted to be mounted on the surface of the wall in an exposed configuration.

The features, functions, and advantages that have been discussed may be achieved independently in various embodiments of the present invention or may be combined with yet other embodiments, further details of which can be seen with reference to the following description and drawings.

### BRIEF DESCRIPTION OF THE DRAWINGS

Having thus described embodiments of the invention in general terms, reference will now be made the accompanying drawings, wherein:

FIG. 1 illustrates a perspective view of the air purifying device, in accordance with an embodiment of the invention;

FIG. 2 illustrates a sectional view of the air purifying device, in accordance with an embodiment of the invention;

FIG. 3 illustrates a side view of the air purifying device, in accordance with an embodiment of the invention;

FIGS. 4A and 4B illustrate a humidifier unit, in accordance with an embodiment of the invention;

FIGS. 5A and 5B illustrate two exemplary mounting options for the air purifying device, in accordance with an embodiment of the invention;

FIG. 6 illustrates an exemplary block diagram of the system environment for air purifying device, in accordance with an embodiment of the invention;

FIG. 7 schematically illustrates an exemplary user device, in accordance with an embodiment of the invention;

FIG. 8 schematically illustrates an access terminal, in accordance with an embodiment of the invention; and

FIG. 9 schematically illustrates a central server system, in accordance with an embodiment of the invention.

### DETAILED DESCRIPTION OF EMBODIMENTS OF THE INVENTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

It should be understood that “operatively coupled,” as used herein, means that the components may be formed integrally with each other, or may be formed separately and coupled together. Furthermore, “operatively coupled” means that the components may be formed directly to each other, or to each other with one or more components located between the components that are operatively coupled together. Furthermore, “operatively coupled” may mean that the components are detachably coupled with each other, or that they are permanently coupled together. In embodiments where the components are detachably coupled, it should be understood that the components are coupled using suitable engagement members, including but not limited to, screws, bolts, adhesive, snap fit, friction fit, magnets, welds, a tongue in groove arrangement, pin, and/or another suitable mechanism. Furthermore, operatively coupled components may mean that the components retain at least some freedom of movement in one or more directions or may be rotated about an axis (i.e., rotationally coupled, pivotally coupled). The engagement members may be made from a suitable material or a combination of materials such as metal, metal alloy, plastic, plastic composite, wood, and/or the like. Furthermore, “operatively coupled” may mean that components may be electronically connected and/or in fluid communication with one another.

It should also be understood that any component, unit, part, or formation described, may have various dimensions. Any dimensional values attributed to a component, unit, part, or formation is to be considered as a particular, non-limiting, embodiment of the invention. Each component, unit, part, or formation may have other values different than the discussed non-limiting embodiment. Those of skill in the art will recognize the various dimensions of any component, unit, part, or formation described herein within the spirit and scope of the presently disclosed.

Pollens, lung damaging dust, smoke, bacteria, viruses, dust mites, and a number of other irritants and microorganisms are commonly found in the ambient air of an enclosed area. It has long been recognized that purification of air can combat some of the effects of airborne particulate contamination. Embodiments of the invention are directed to an air purifying device for filtering airborne contaminate particles and for sterilizing the air by means of ultraviolet radiation. To this end, the invention provides an improved configuration and method which enhances the efficiency of filtering airborne contaminate particles in an enclosed area. Moreover, the self-contained construction of the air purifying device allows for relatively effortless installation and operation as a wall-mounted unit that may be powered using any conventional power source. In one embodiment, the air purifying device is powered by an alternating current (AC) power supply (e.g., connection to a wall outlet). In another embodiment, the air purifying device 100 may be alternatively or additionally powered through a direct current (DC) power supply by way of an internal battery that functions as an auxiliary or emergency power supply should a primary power supply fail (e.g., power outage).

Air Purifying Device Construction

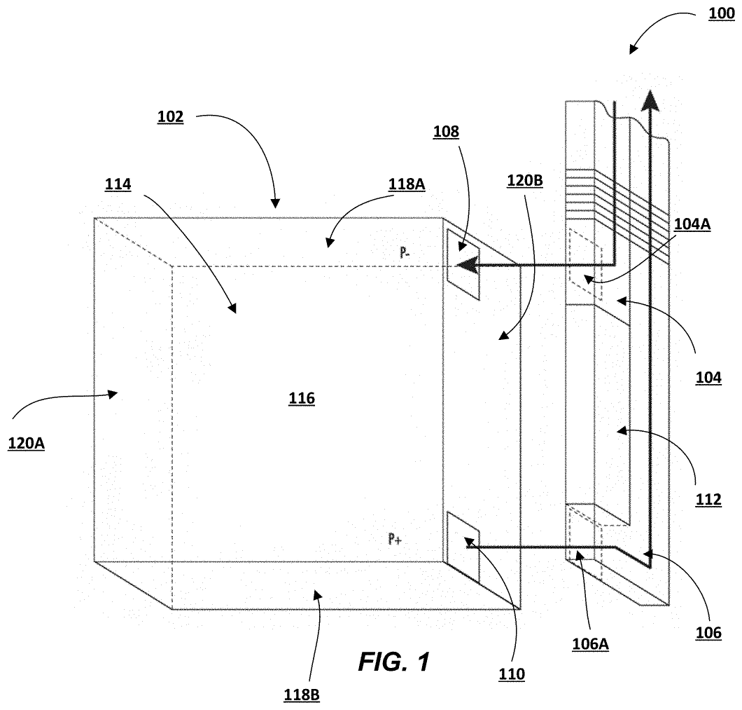

FIG. 1 illustrates a perspective view of the air purifying device 100, in accordance with an embodiment of the invention. As illustrated in FIG. 1, the air purifying device 100 includes a housing or housing frame 102 that collects, supports, and/or houses one or more units of the air purifying device 100 described herein. The housing 102 has may include a first wall 114 and an opposite second wall 116. The first wall 114 and the opposite second wall 116 are terminated and separated by a first pair of opposing lateral walls 118A and 118B, and a second pair of opposing lateral walls 120A and 120B), as best illustrated in FIG. 1. The housing 102 may include an inlet aperture 108—an opening, structure, or system—formed on the lateral wall 120B, through which unfiltered air is received into the air purifying device 100. The housing 102 may also include an outlet aperture 110—an opening, structure, or system—formed on the lateral wall 120B, through which filtered air is exhausted from the air purifying device 100. In a particular, non-limiting embodiment, the air purifying device 100 and housing 102 has an overall height, width, and depth of approximately 36 inches×36 inches×10 inches, respectively. Also, in a particular, non-limiting, embodiment, the inlet aperture 108 and outlet aperture 110 each have an overall height, and width of approximately 5 inches×4 inches.

As illustrated in FIG. 1, the air purifying device 100 may include an inlet air distributor 104 that is operatively coupled to the housing 102. In one embodiment, a first end of the inlet air distributor 104 is adapted to be detachably coupled to the housing 102 proximate to the inlet aperture 108 and is configured to direct air into the inlet aperture 108 for treatment. In one aspect, the inlet air distributor 104 may have an intake opening 104A formed proximate to the first end and having a dimension that is substantially similar to that of the inlet aperture 108. The intake opening 104A is formed in such a way that when the inlet air distributor 104 is in a coupled position with the housing 102, the inlet aperture 108 and the intake opening 104A overlap to allow air flow from the inlet air distributor 104 into the air purifying device 100.

In some embodiments, a second end of the inlet air distributor may be configured to be operatively coupled to an existing heating, ventilation, and air conditioning (HVAC) system installed to provide environmental comfort in the enclosed area. In one aspect, the inlet air distributor is configured to be operatively coupled to a supply air duct of the HVAC system. In another aspect, the inlet air distributor is configured to operatively be coupled to a return air duct of the HVAC system. In some other embodiments, the second end of the inlet air distributor may be configured to draw air from an enclosed area (e.g., room, hallway, corridor) rendering the air purifying device 100 to be truly stand-alone in its configuration. In this regard, the second end of the inlet air distributor 104 may be configured to draw air from the upper strata of the enclosed area (e.g., proximate to the ceiling). In its stand-alone configuration, the air purifying device 100 removes contaminated air from the enclosed area independently of the common air mass in circulation from the existing HVAC system via the inlet air distributor 104 by creating a negative pressure channel (as described in further detail below). In one aspect, the second end of the inlet air distributor 104 may be operatively coupled to a perforated cover, such as a grille, register, vent, and/or the like to draw air from the ambient air in the enclosed area into the inlet air distributor 104.

As illustrated in FIG. 1, the air purifying device 100 may include an outlet air distributor 106 that is operatively coupled to the housing 102. In one embodiment, a first end of the outlet air distributor 106 is adapted to be detachably coupled to the housing 102 proximate to the outlet aperture 110 and is configured to direct filtered air received from the outlet aperture 110. In one aspect, the outlet air distributor 104 may have an exhaust opening 106A formed proximate to the first end and having a dimension that is substantially similar to that of the outlet aperture 108. The exhaust opening 104A is formed in such a way that when the outlet air distributor 106 is in a coupled position with the housing 102, the outlet aperture 108 and the exhaust opening 106A overlap to allow air flow from the air purifying device 100 into the outlet air distributor 106.

In some embodiments, a second end of the outlet air distributor 106 may be configured to be operatively coupled to an existing HVAC system. In one aspect, the outlet air distributor is configured to be operatively coupled to a supply air duct of the HVAC system. In another aspect, the outlet air distributor is configured to operatively be coupled to a return air duct of the HVAC system. In some other embodiments, the second end of the outlet air distributor 106 may be configured to exhaust the filtered air directly into an enclosed area (e.g., room, hallway, corridor). In this regard, the second end of the outlet air distributor 106 may be configured to deliver filtered air into the lower strata of the enclosed area (e.g., proximate to the floor). In such a stand-alone configuration, the air purifying device 100 delivers filtered air from the enclosed area independently of the common air mass in circulation from the existing HVAC system via the outlet air distributor 106 by creating a positive pressure channel (as described in further detail below). In one aspect, the second end of the outlet air distributor 106 may be operatively coupled to a perforated cover, such as a grille, register, vent, and/or the like to exhaust the filtered air directly into the ambient air in the enclosed area.

In some embodiments, the inlet air distributor 104 and the outlet air distributor 106 may be operatively coupled to each other, as illustrated in FIG. 1. In one aspect, the inlet air distributor 104 and the outlet air distributor 106 may be operative coupled to each other in an adjoining configuration where they extend alongside each other in a parallel arrangement, a vertical arrangement, or any other applicable arrangement. Those of skill in the art will recognize the various types of air distributor ducting configuration and/or arrangements that may be used within the spirit and scope of the presently disclosed. In a particular, non-limiting embodiment, the inlet air distributor 104 and the outlet air distributor 106 may have an overall width and depth of approximately 5 inches×3 inches, respectively with a length varying based on the mounting configuration of the air purifying device 100.

As illustrated in FIG. 1, the air purifying device 100 may include an access terminal 112 that is operatively coupled to the lateral wall 120B of the housing 102. The access terminal 112 is positioned to be accessible and is configured for receiving and transmitting instructions for operating or controlling the one or more units of the air purifying device 100 such as the primary filtration unit, the secondary filtration unit, the particulate filtration unit, and/or the humidifier unit. The air purifying device 100 may be actuated using one or more control options available via the access terminal 112. During operation, the access terminal 112 may be used to control functionality of the one or more units and its components of the air purifying device 100. For example, the access terminal 112 may be used to establish a preset flow rate (described in further detail herein) for the flow rate sensors, set a timing window during which the air purifying device 100 will remain operational (after which it will automatically turn off), and/or the like. The access terminal 112 may also be configured to display a real-time operational status, state, condition, or situation of each component associated with the one or more units of the air purifying device 100. For example, the access terminal 112 may be configured to display the status of the air filters (such as the high efficiency particulate air (HEPA) filter and the ultra-low particle air (ULPA) filter) used in each unit, status of the plurality of ultraviolet light sources used in each unit, status of the air regulation devices used in each unit, and/or the like. The access terminal 112 may also be configured to display alerts to communicate any operational issues associated with the one or more units and its components. For example, the access terminal 112 may be configured to display an alert identifying any malfunctioning component associated with the one or more units, alert indicating filter replacement requirement, and/or the like based on measured output readings from the one or more sensors (such as the air quality sensor) in the one or more units.

In some embodiments, the access terminal 112 is configured to be in network communication with at least the user device and/or the central server. By way of this network communication, the user device and/or the central server may be used, in remote operation, to control the actuation of, operational functionality of, display operational status of, and/or display alerts related to the one or more units and its associated components (described in further detail herein).

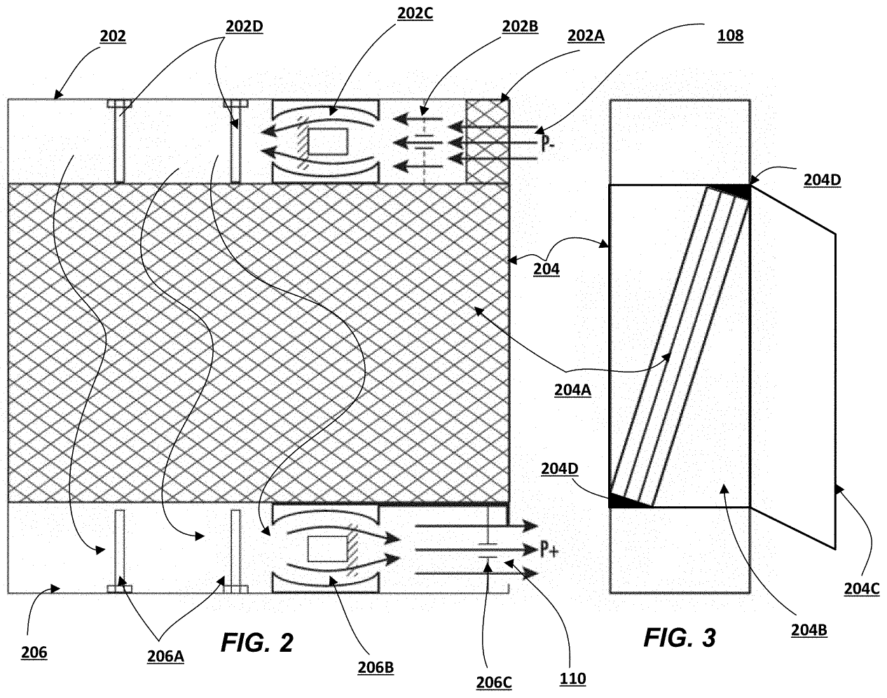

FIG. 2 illustrates a sectional side view of the air purifying device 200, in accordance with an embodiment of the invention. As illustrated in FIG. 2, the air purifying device 100 includes a primary filtration unit 202, a particulate filtration unit 204, and a secondary filtration unit 206. In one embodiment, the primary filtration unit 202, the particulate filtration unit 204, and the secondary filtration unit 206 are positioned within the housing 102. In this regard, the primary filtration unit 202 is positioned proximate to the inlet aperture 108 and is configured to receive air through the inlet aperture 108 for treatment. The secondary filtration unit 206 is positioned proximate to the outlet aperture 110 and is configured to exhaust the filtered air through the outlet aperture 110. In one embodiment, the particulate filtration unit 204 is positioned between the primary filtration unit 202 and the secondary filtration unit 206 as illustrated in FIG. 2, providing an airflow pathway for the flow of air from the primary filtration unit 202 to the secondary filtration unit 206 through the particulate filtration unit 204.

As illustrated in FIG. 2, the primary filtration unit 202 may include a high efficiency particulate air (HEPA) filter 202A, an inlet airflow sensor 202B, an airflow regulation device 202C, and a plurality of ultraviolet light sources 202D. As illustrated in FIG. 2, the high efficiency particulate air (HEPA) filter 202A may be positioned proximate to the inlet aperture 108 and configured to pre-filter airborne particulate matter from the air received from the inlet aperture 108. In one embodiment, the primary filtration unit 202 may include a filter aperture formed therein and configured to have any suitable geometry to receive and house the high efficiency particulate air (HEPA) filter 202A. The high efficiency particulate air (HEPA) filter 202A may be received into the filter aperture in any suitable orientation, such as along vertical plane, along a horizontal plane, or at any angle relative to the vertical or horizontal plane. In one embodiment, the high efficiency particulate air (HEPA) filter may be configured to pre-filter airborne particulate matter of a size equal to or greater than 0.3 micron from the air received from the inlet aperture 108, and to a lesser degree, pre-filter airborne particulate matter of a size less than 0.3 micron from diffusion of air received from the inlet aperture. As illustrated in FIG. 2, the inlet airflow sensor 202B may be positioned proximate to the high efficiency particulate air (HEPA) filter 202A and configured to receive air filtered by the high efficiency particulate air (HEPA) filter 202A. In one embodiment, the inlet airflow sensor 202B may be configured to measure a flow rate of air filtered by the high efficiency particulate air (HEPA) filter 202A. As illustrated in FIG. 2, the airflow regulation device 202C may be configured to regulate and direct the flow of air filtered by the high efficiency particulate air (HEPA) filter 202A towards the plurality of ultraviolet light sources 202D based on at least the flow rate of air measured by the inlet airflow sensor 202B. In embodiments contemplated herein, the airflow regulation device 202C may be adapted to accept air from a first point and transport it to a second point. In this regard, the airflow regulation device 202C may be a fan; however, fan alternatives, e.g., pumps, variable apertures, removeable panels, etc., may be used to achieve a similar result. As illustrated in FIG. 2, the plurality of ultraviolet light sources 202D is adapted to be positioned longitudinally within the primary filtration unit 202 and configured to eliminate pathogens such as bacteria, mold spores, fungi, viruses, as well as other microorganisms by exposing the air to short wave ultraviolet radiation (UV-C). In other embodiments, the plurality of ultraviolet light sources 202D could be positioned in a different orientation, such as laterally, within the primary filtration unit 202. In a particular, non-limiting embodiment, the primary filtration unit 202 has an overall height, width, and depth of approximately 36 inches×6 inches×10 inches, respectively. As those of the skill of the art will recognize, in other embodiments, primary filtration unit 202 could contain different quantities of high efficiency particulate air (HEPA) filters, airflow regulation devices, ultraviolet light sources, and/or sensors, and the order in which they are arranged along the airflow in primary filtration unit 202 could be changed.

Air filters, such as the high efficiency particulate filter 202A, that are able to filter airborne particulate matters up to 0.3 micron in size tend to create resistance to air flow by design, causing a high differential pressure drop across the filter. In addition, over time, the air filters may collect particulate matter on the surface thereon, further restricting the flow of air thereacross. The airflow sensor 202B may be configured to sense the change in pressure (total pressure and static pressure) that is produced through the movement of air (or lack thereof). To compensate for any change in pressure that occurs due to the use of the high efficiency particulate air (HEPA) filter 202A, and to maintain a preset flow rate of air within the primary filtration unit 202, the airflow regulation device 202C is employed. In one embodiment, the airflow regulation device 202C may be configured to have sufficient capacity to maintain the flow rate of air at maximum differential pressure under which the air purifying device 100 could operate. To this end, the airflow regulation device 202C may be configured to be automatically actuated based on the preset flow rate of air and the flow rate of air measured by the airflow sensor 202B. In one embodiment, if the flow rate of air measured by the airflow sensor 202B is less than the preset flow rate of air, the airflow regulation device 202C may be automatically actuated causing negative air pressure, and forcing a larger volume of air to flow into the primary filtration unit 202 via the inlet aperture 108. This may be due to the natural design of the high efficiency particulate air (HEPA) filter 202A that creates air resistance, due to aggregate particulate matter collected and formed on the surface of the high efficiency particulate air (HEPA) filter 202A over time, and/or the like. When the flow rate of air measured by the airflow sensor 202B is equal to or greater than the preset flow rate of air indicating a steady air flow, the airflow regulation device 202C may be automatically turned off. In another embodiment, the airflow regulation device 202C may be configured to operate continuously, creating a continuous negative air pressure channel within the inlet air distributor 104, that results in extraction of air from the enclosed area, through the inlet air distributor 104, and into the primary filtration unit 202 via the inlet aperture 108. In instances where the flow rate of air measured by the airflow sensor 202B drops below a preset flow rate of air, the speed of the airflow regulation device 202C may be configured to automatically increase to allow for a larger volume of air to be drawn in through the inlet aperture 108 until the measured flow rate of air meets the preset flow rate. In some embodiments, the flow rate of air in the primary filtration unit 202 may be preset and monitored using the access terminal 112 (described in further detail herein).

The air displaced by the airflow regulation device 202C is then exposed to the plurality of ultraviolet light sources 202D and passed therethrough. The plurality of ultraviolet light sources 202D is configured to generate short wave ultraviolet radiation (UV-C) to treat the air filtered by the high efficiency particulate air (HEPA) filter 202A and displaced by the airflow regulation device 202C. By exposing the air to UV-C radiation, the plurality of ultraviolet light sources 202D are designed to change the DNA and RNA of bacteria and viruses (such as the sars-COV-2), destroying their ability to reproduce.

As illustrated in FIG. 2, the air purifying device 100 may include a particulate filtration unit 204 having an aperture that is capable of housing an ultra-low particle (ULPA) filter 204A therein. The ultra-low particle (ULPA) filter 204A may be received into the aperture in any suitable orientation, such as along vertical plane, along a horizontal plane, or at any angle relative to the vertical or horizontal plane. In one embodiment, when the air is moved from the primary filtration unit 202 to the particulate filtration unit 204 via the airflow pathway, the ultra-low particle air (ULPA) filter 204A housed within the particulate filtration unit 204 is configured to filter any lingering airborne particulate matter of a size equal to or greater than 0.12 micron from the air received from the primary filtration unit 202, and to a lesser degree, smaller airborne particulate matter through diffusion. In a particular, non-limiting embodiment, the particulate filtration unit 204 has an overall height, width, and depth of approximately 36 inches×24 inches×10 inches, respectively.

As illustrated in FIG. 2, the secondary filtration unit 206 may include a plurality of ultraviolet light sources 206A, an airflow regulation device 206B, and an outlet airflow sensor 206C. In one embodiment, the plurality of ultraviolet light sources 206A is adapted to be positioned longitudinally within the secondary filtration unit 206. Similar to the plurality of ultraviolet light sources 202D in the primary filtration unit 202, the plurality of ultraviolet light sources 206A is configured to eliminate any lingering pathogens in the air previously irradiated by the plurality of ultraviolet light sources 202D, by exposing the air to short wave ultraviolet radiation (UV-C). In other embodiments, the plurality of ultraviolet light sources 206A could be positioned in a different orientation, such as laterally within the secondary filtration unit 206. As illustrated in FIG. 2, the airflow regulation device 206B may be configured to direct and control the flow of air from the plurality of ultraviolet light sources 206D towards the outlet airflow sensor 206C and the outlet aperture 110. In one embodiment, the airflow regulation device 206B may be configured to direct the flow of air received from the plurality of ultraviolet light sources 206D having passed through the ultra-low particulate air (ULPA) filter 204A, towards the outlet aperture 110 based on at least the flow rate of the air measured by the outlet airflow sensor 206C. Similar to the airflow regulation device 202C, the airflow regulation device 206B may be adapted to accept air from a first point and transport it to a second point, and may be fan, or fan alternative. As illustrated in FIG. 2, the outlet airflow sensor 206C may be positioned proximate to the outlet aperture 110 and configured to receive air from the airflow regulation device 206B. In one embodiment, the outlet airflow sensor 206C may be configured to measure the flow rate of air received from the airflow regulation device 206B. In one embodiment, the secondary filtration unit 206 may include an air quality sensor (not shown). The air quality sensor may be configured to continuously sample the filtered air to detect particulate contamination, gas contamination, and/or the like. In one embodiment, by continuously sampling the filtered air, the readings from the air quality sensor may be used to determine the need for servicing and/or replacement the plurality of ultraviolet light sources, airflow regulation devices, and/or the air filters used in the one or more units. In a particular, non-limiting embodiment, the secondary filtration unit 206 has an overall height, width, and depth of approximately 36 inches×6 inches×10 inches, respectively. As those of the skill of the art will recognize, in other embodiments, secondary filtration unit 206 could contain different quantities of ultraviolet light sources, air regulation devices, and/or sensors, and the order in which they are arranged along the airflow in secondary filtration unit 206 could be changed.

Air filters, such as the ultra-low particle air (ULPA) filter 204A, that are able to filter out airborne particulate matters up to 0.12 micron in size have a filter media that are dense, reducing their capacity to move air through them. As a result, air circulation tends to be lower in enclosed areas where air is treated using ULPA filter-based air treatment devices as the air exhausting from such air treatment devices have a low flow rate. To address this issue, the outlet airflow sensor 206C may be configured to continuously measure the flow rate of air received from the airflow regulation device 206B, and similar to the primary filtration unit 202, automatically actuate the airflow regulation device 206B based on a preset flow rate of air. In one embodiment, when the flow rate of air measured by the outlet airflow sensor 206C is less than the preset flow rate of air, the airflow regulation device 206B may be automatically actuated to move a larger volume of air from the plurality of light sources 206A towards the outlet aperture 110 by creating a positive pressure differential across the airflow regulation device 206B. When the flow rate of air measured by the airflow sensor 206C is equal to the preset flow rate of air, the airflow regulation device 206B may be automatically turned off. This is done to ensure that the enclosed area is provided with a sufficient volume of filtered air. In another embodiment, the airflow regulation device 202C may be configured to operate continuously, creating a continuous positive air pressure channel within the inlet air distributor 104, that results in extraction of air from the enclosed area, through the inlet air distributor 104, and into the primary filtration unit 202 via the inlet aperture 108. In instances where the flow rate of air measured by the airflow sensor 202B drops below a preset flow rate of air, the speed of the airflow regulation device 202C may be configured to automatically increase to allow for a larger volume of air to be drawn in through the inlet aperture 108 until the measured flow rate of air meets the preset flow rate. In some embodiments, the flow rate of air in the secondary filtration unit 206 may be preset and monitored using the access terminal 112 (described in further detail herein).

In one embodiment, the secondary filtration unit 206 may include a high efficiency gas absorption (HEGA) filter. The high efficiency gas absorption (HEGA) filter may be positioned proximate to the outlet aperture 110 and configured to absorb polluting odors, gases, and volatile organic compounds (VOCs) from the air received from the airflow regulation device 206B. In one embodiment, the secondary filtration unit 206 may include a filter aperture formed therein and configured to have any suitable geometry to receive and house the high efficiency gas absorption (HEGA) filter. The high efficiency gas absorption (HEGA) may be received into the filter aperture in any suitable orientation, such as along vertical plane, along a horizontal plane, or at any angle relative to the vertical or horizontal plane.

As described herein, the air entering the air purifying device 100 for treatment is filtered and/or radiated to remove airborne particulates and pathogens at each of the primary filtration unit 202, the particulate filtration unit 204, and the secondary filtration unit 206. First, the air is filtered and treated by primary filtration unit 202. Then, the air, having gone through a first stage of treatment (at the primary filtration unit 202), is then pushed through the airflow pathway into the particulate filtration unit 204 where it is filtered further by the ultra-low particle air (ULPA) filter 204A. Then, the air, having gone through the first stage of treatment (at the primary filtration unit 202) and a second stage of treatment (at the particulate filtration unit 204), is then pushed through the airflow pathway into the secondary filtration unit 206 where it undergoes a last stage of treatment, before being delivered through the outlet aperture 110 to the outlet air distributor 106.

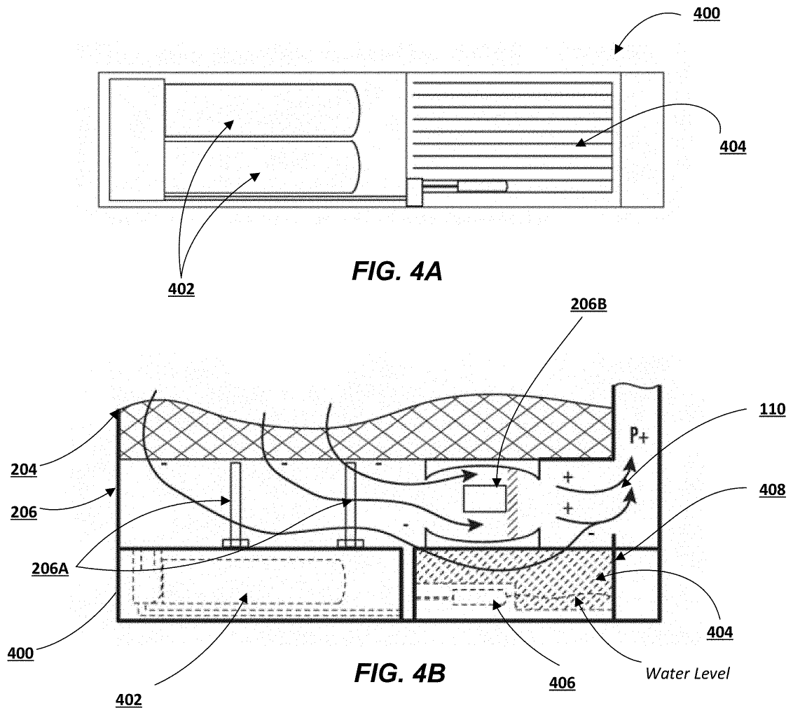

FIG. 3 illustrates a side view of the air purifying device 100, in accordance with an embodiment of the invention. As illustrated in FIG. 3, the particulate filtration unit 204 includes an aperture that is capable of slidably receiving an ultra-low particle (ULPA) filter 204A via an access opening 204B. The access opening 204B provides access to the ultra-low particle air (ULPA) filter 204A positioned within the aperture of the particulate filtration unit 204 for purposes of inspection and replacement of the filter. In one embodiment, the particulate filtration unit 204 may also include an air filter access door 204C that is operatively coupled to the lateral wall 120B of the housing 102 and is provided to cover and seal the access opening to prevent air from escaping the air purifying device 100 through the access opening 204B. As illustrated in FIG. 3, the particulate filtration unit includes a plurality of stabilizing elements 204D arranged on an interior wall of the aperture to slidably receive the ultra-low particle air (ULPA) filter 204A such that when the ultra-low particle air (ULPA) filter 204A is inserted into the aperture of particulate filtration unit 204 through the access opening 204B, the plurality of stabilizing elements 204D form a secure seal with at least a top edge and a bottom edge of the ultra-low particle air (ULPA) filter 204A to secure the ultra-low particle air (ULPA) filter 204A.

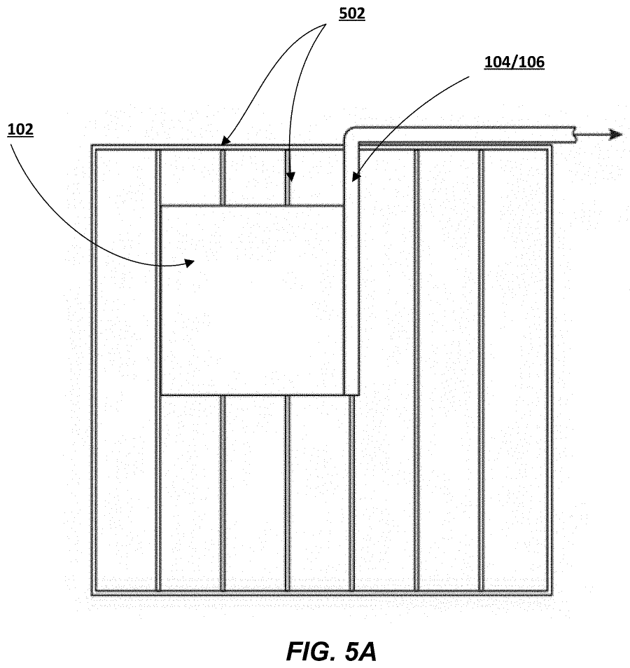

FIGS. 4A and 4B illustrate a humidifier unit 400, in accordance with an embodiment of the invention. More particularly, FIG. 4A illustrates a sectional top view of a humidifier unit 400, and FIG. 4B illustrates a half sectional side view of the air purifying device 100 with the humidifier unit 400. As illustrated in FIG. 4A, the humidifier unit 400 includes a plurality of water filters 402, a water basin 408, a float valve 406, and a wick cartridge 404. In one embodiment, the plurality of water filters 402 may be configured to remove waterborne contaminate matter from water received from a water source to produce filtered water. The filtered water filtered by the plurality of water filters 402 is then received by and stored in the water basin 408. The float valve 406 is positioned within the water basin 408 and is configured to measure the water level in the water basin 408. The wick cartridge 404 is configured to absorb the filtered water from the water basin 408.

As illustrated in FIG. 4B, the optional humidifier unit 400 is configured to be operatively coupled to the secondary filtration unit 206 of the air purifying device 100 to add water or humidity to the filtered air. When coupled, in some embodiments, the secondary filtration unit 206 and the humidifier unit 404 may form a conduit or passage to facilitate the flow of air from the secondary filtration unit 206 to the humidifier unit 400. Also, when coupled, in other embodiments, the secondary filtration unit 206 and the humidifier unit 400 may be adapted to form one or more access openings to allow wick cartridge 404 to move from the humidifier unit 400 into the secondary filtration unit 206 and into the path of the filtered air. During normal operation, the conduit, and the access openings between the secondary filtration unit 206 and the humidifier unit 400 may be adapted to remain in a closed configuration allowing the filtered air in the secondary filtration unit 206 to be directed towards the outlet aperture 110.

The actuation of the humidifier unit 404 may be initiated based on the measured moisture content in the filtered air before being exhaust through the outlet aperture 110. Accordingly, the second filtration unit 206 may include a humidity and temperature sensor (not shown) to measure the moisture content in the filtered air before being dispersed through the outlet aperture 110. Should the moisture content drop below a preset moisture level, in one embodiment, the humidifier unit 400 is automatically actuated and the filtered air, before being dispersed through the outlet aperture 110, is treated by the humidifier unit 400 to increase the concentration of water vapor therein.

In one embodiment, to increase the concentration of water vapor in the air, the air received from the plurality of light sources 206A is passed through the water soaked wick cartridge 404 set in the water basin 408. To achieve this, in one aspect, when the humidifier unit 400 is actuated, the conduit between the secondary filtration unit 206 and the humidifier unit 400 may be configured to automatically open, to facilitate and direct the flow of filtered air from the secondary filtration unit 206 into the humidifier unit 400 and through the wick cartridge 404 positioned therein. In another aspect, to increase the concentration of water vapor in the air, the wick cartridge 404 having absorbed water from the water basin 408 may be adapted to move into the path of the filtered air in the secondary filtration unit 206, via the access openings, without requiring the filtered air to be redirected.

When the filtered air is passed through the wick cartridge 404, the wick cartridge 404 infuses moisture into the air. This process gradually desiccates the wick cartridge 404 causing the wick cartridge 404 to then absorb more filtered water from the water basin 408. To provide for this constant absorption, the float valve 406 is configured to measure the water level in the water basin 408. Each time the wick cartridge 404 absorbs more water from the water basin 408, the water level in the water basin 408 drops. This drop is measured by the float valve 406 which automatically actuates the water refilling process to refill the water basin 408 with filtered water received from the plurality of water filters 402. This process is repeated until the moisture content in the air meets the present moisture level. When this happens, the humidifier unit 400 is automatically turned off, the conduit and/or the access openings automatically revert(s) to its closed configuration, and the filtered air is exhausted through the outlet aperture 110 without being passed through the humidifier unit 400.

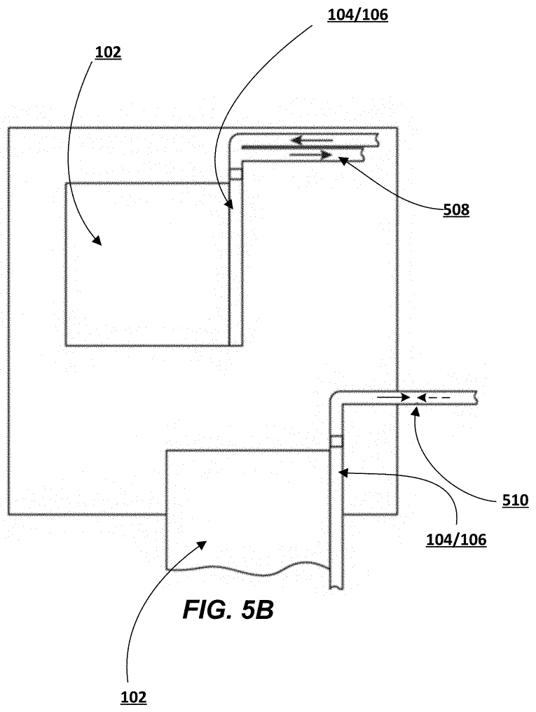

FIGS. 5A and 5B illustrate two exemplary mounting options for the air purifying device, in accordance with an embodiment of the invention. As shown in FIG. 5A, the housing 102 may be adapted to be mounted in line with or intermediate of a plurality of framing members 502 of a wall in a concealed configuration. As illustrated in FIG. 5B, the housing 102 may be adapted to be mounted on the surface of the wall in an exposed configuration. In either or both mounting options, the opposite second wall 116 of the housing 102 may be configured to include a plurality of mounting members capable of mounting the housing 102 either in line with or intermediate of a plurality of framing members of a wall, or on the surface of the wall itself. Mounting members or wall mounts may include, but are not limited to clips, brackets, screws, connectors, or extrusions. Those of skill in the art will recognize the various types of extrusions that may be used within the spirit and scope of the presently disclosed.

Once mounted, the air distributors 104 and 106 may be operatively coupled to dependent ducting to direct the flow of air to and from the air purifying device 100. In some embodiments, the air purifying device 100 may be adapted to operate in a stand-alone configuration in an environment with multiple enclosed areas where an existing HVAC system is employed to circulate common air mass. In such configurations, the inlet air distributor 104 may be operatively coupled to dependent ducting that may extend into each enclosed area. The dependent ducting may be operatively coupled to a perforated cover, such as a grille, register, vent, and/or the like adapted to draw air into the air purifying device 100, from the enclosed area, by creating a negative pressure differential. The outlet air distributor 106 may also be operatively coupled to dependent ducting that may extend into each enclosed area. Similarly, the dependent ducting may be operatively coupled to a perforated cover, such as a grille, register, vent, and/or the like adapted to direct filtered air, from the air purifying device 100, into the enclosed areas by creating a positive pressure differential. In one embodiment, the filtered air that is exhausted by the air purifying device 100 and distributed by the outlet air distributor 106 via dependent ducting into the enclosed areas may be drawn in by the existing HVAC system for heating and cooling purposes. The existing HVAC system uses the drawn in filtered air for processing and distributes the filtered and processed air into the enclosed areas via HVAC ducting. By continuously operating the air purifying device 100 with existing HVAC systems in this manner, the filtered air exhausted by the air purifying device 100 may gradually replace the common air mass circulated by the HVAC system, resulting in a series of “clean” enclosed areas.

In this regard, the dependent ducting coupled to the air distributors 104 and 106 may be configured for installation in an adjoining configuration extending adjacent to each other, as shown in FIG. 5B. In one embodiment, the dependent ducting may be adapted to extend adjacent to each other in a parallel arrangement 510. In another embodiment, the dependent ducting may be adapted to extend adjacent to each other in a vertical arrangement 508 where they are stacked on one another as they extend from the air distributors 104 and 106. Those of skill in the art will recognize the various types of air distributor ducting configuration and/or arrangements that may be used within the spirit and scope of the presently disclosed.

In one embodiment, each surface (e.g., interior surface) of the air purifying device 100, including the surfaces of the primary filtration unit 202, the particulate filtration unit 204, the secondary filtration unit 206, the humidifier unit 400, the inlet air distributors 104 and 106, and any dependent ducting may be deposited with antibacterial coating (e.g., passive bacteriostatic coating) to hinder bacterial attachment on surfaces and/or to kill bacteria upon contact with the surfaces.

It is to be understood that the air filters described herein, specifically the high efficiency particulate air (HEPA) filter and the ultra-low particle air (ULPA) filters are exemplary, and those of skill in the art will recognize that any air filters that may be used within the spirit and scope of the presently disclosed.

### Air Purifying Device System Environment and Operation

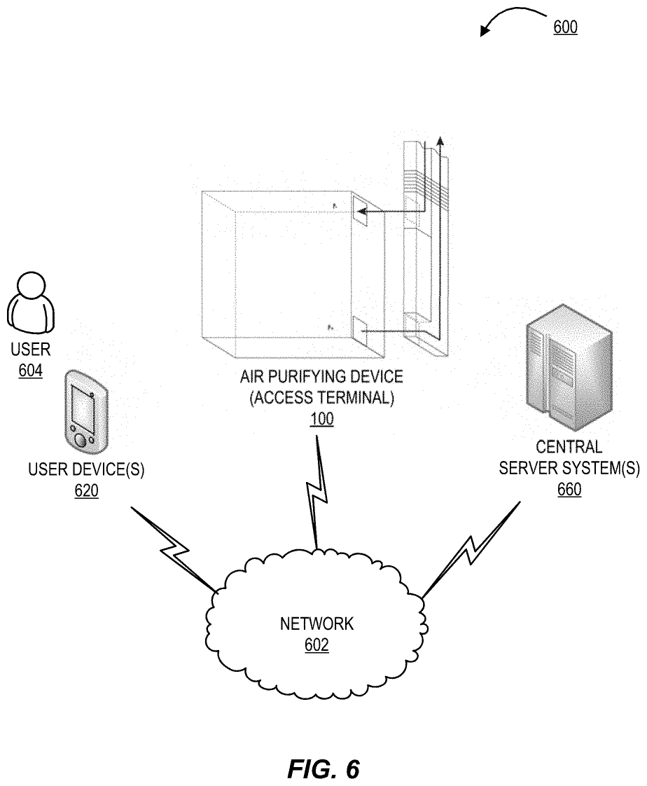

FIG. 6 presents an exemplary block diagram of the system environment for air purifying device 600, in accordance with an embodiment of the invention. The environment 600 comprises a user device 620 associated with a user 604, the air purifying device 100 (more specifically, the access terminal 112 of the air purifying device 100), and a central server system 660 that may be used to allow for remote operational control of the air purifying device 100. To this end, the user device 620 and/or the central server system 660 may be used to control the actuation of, operational functionality of, display operational status of, and/or display alerts related to the one or more units and its associated components via the network 602. In embodiments contemplated herein, the system environment 600 may include a plurality of air purifying devices, where each of the plurality of air purifying devices (more specifically, the access terminals in each of the plurality of air purifying devices) are connected to each other, to the user device 620, and/or to the central server system 660 via the network 602. This allows for a user 604 to control the actuation of, operational functionality of, display operational status of, and/or display alerts related to the one or more units and its associated components for each of the plurality of air purifying devices, via the network 602.

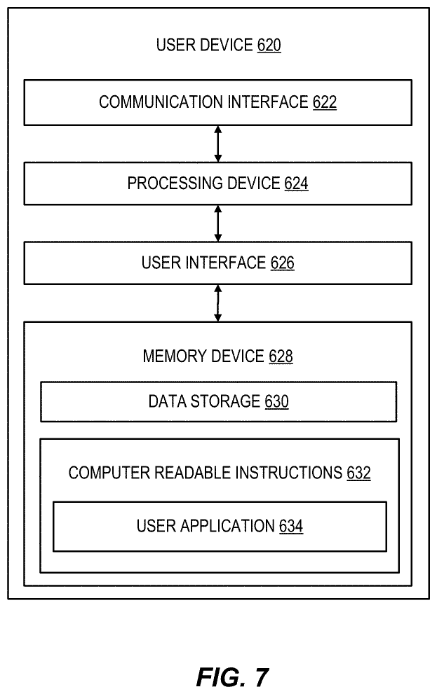

As used herein, a “processing device,” such as the processing devices 624, 644, and 664 (described with respect to FIGS. 7-9, respectively), generally refers to a device or combination of devices having circuitry used for implementing the communication and/or logic functions of a particular system. For example, a processing device may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processing device may further include functionality to operate one or more software programs based on computer-executable program code thereof, which may be stored in a memory. As the phrase is used herein, a processing device may be “configured to” perform a certain function in a variety of ways, including, for example, by having one or more general-purpose circuits perform the function by executing particular computer-executable program code embodied in computer-readable medium, and/or by having one or more application-specific circuits perform the function.

As described herein, a “user” may be an individual associated with the air purifying device. As such, in some embodiments, the user may be an operator of a user application (such as the air purifying device application 656) that is configured to control the operation of the air purifying device 100 and/or an operator of the access terminal 112 positioned on the air purifying device 100 that controls the operation of the air purifying device 100.

As used herein, “authentication credentials” may be any information that can be used to identify of a user allowing for authentication of a user requesting access to the air purifying device 100. For example, a system may prompt a user to enter authentication information such as a username, a password, a personal identification number (PIN), a passcode, biometric information (e.g., iris recognition, retina scans, fingerprints, finger veins, palm veins, palm prints, digital bone anatomy/structure and positioning (distal phalanges, intermediate phalanges, proximal phalanges, and the like), an answer to a security question, a unique intrinsic user activity, such as making a predefined motion with a user device. This authentication information may be used to authenticate the identity of the user (e.g., determine that the authentication information is associated with the account) and determine that the user has authority to operate the air purifying device.

As used herein, a “user interface,” such as the user interfaces 626, 646, and 666 (described with respect to FIGS. 7-9, respectively), generally includes a plurality of interface devices and/or software that allow a user to input commands and data to direct the processing device to execute instructions. For example, a user interface may include a graphical user interface (GUI) or an interface to input computer-executable instructions that direct the processing device to carry out specific functions. The user interface employs certain input and output devices to input data received from a user or output data to a user. These input and output devices may include a display, mouse, keyboard, button, touchpad, touch screen, microphone, speaker, LED, light, joystick, switch, buzzer, bell, and/or the like.

As used herein, a “memory device,” such as memory devices 628, 648, and 668 (described with respect to FIGS. 7-9, respectively), generally refers to a device or combination of devices that store one or more forms of computer-readable media for storing data and/or computer-executable program code/instructions. Computer-readable media is defined in greater detail below. For example, in one embodiment, the memory device includes any computer memory that provides an actual or virtual space to store data temporarily or permanently and/or commands provided to the processing device when it carries out its functions described herein.

As used herein, a “communication interface,” such as communication interfaces 622, 642, and 662 (described with respect to FIGS. 7-9, respectively), generally includes a modem, server, transceiver, and/or other device for communicating with other devices on a network, and/or a user interface for communicating with the user directly. A communication interface may have one or more communication devices configured to communicate with one or more other devices on a network, such as a user device, computer system, server system, cloud server system, and/or the like. The processing device is configured to use the network communication interface to transmit and/or receive data and/or commands to and/or from the other devices connected to the network.

The systems and devices communicate with one another over the network 602 via one or more communication channels and perform one or more of the various steps and/or methods according to embodiments of the disclosure discussed herein. The network 602 and the one or more communication channels may include a local area network (LAN), a wide area network (WAN), and/or a global area network (GAN). The network 602 may provide for wireline, wireless, or a combination of wireline and wireless communication between devices in the network. In one embodiment, the network 602 includes the Internet. In some embodiments, the network 602 includes wireless communication, such as near field communication. The one or more communication channels allow the various systems of the environment to transmit and receive data, control signals, and commands to and from one another.

Referring now to FIG. 7, which schematically depicts a user device, in accordance with one embodiment of the invention, allows the user to transmit and/or receive information or commands to and from the air purifying device 100 and/or the central server system 660 via the network 602. Any communication between the user device 620 and the air purifying device 100 and/or the central server system 660 (or any other networked device) may be subject to an authentication protocol allowing the central server system 660 to maintain security by permitting only authenticated users (or processes) to access the air purifying device 100 for operation, control, and/or the like. In this regard, the user device 620 may be configured to require the user to provide authentication credentials to determine whether the user is eligible to access the air purifying device 100 and/or the central server system 660.

As illustrated in FIG. 7, the user device 620 includes a communication interface 622 communicably coupled with a processing device 624, which is also communicably coupled with a memory device 628. In some embodiments, the communication interface 622 may also comprise a GPS transceiver capable of determining a geographic location associated with the user device 620. The processing device 624 is configured to control the communication interface 622 such that the user device 620 communicates across the network 602 with the air purifying device 100, the central server system 660, and/or one or more other systems. The processing device 624 is also configured to access the memory device 628 in order to read the computer readable instructions 632, which in some embodiments includes a user application 634. The user application 634 allows for communication of the user device 620 with the other systems and devices within the environment 600 such as the central server system 660. As described herein, the user application 634 allows the user 604 to receive information from as well as transmit information to other systems and communicate with entities and third parties within the system environment 600. The memory device 628 also includes a data storage or repository 630 or similar storage device for storing pieces of data (e.g., user identifying authentication information or credentials) that can be accessed by the processing device 624. In accordance with embodiments of the invention, the user device is intended to represent various forms of mobile devices, such as personal digital assistants, cellular telephones, smartphones, and other similar computing devices. The components shown here, their connections and relationships, and their functions, are meant to be exemplary only, and are not meant to limit implementations of the inventions described and/or claimed in this document.

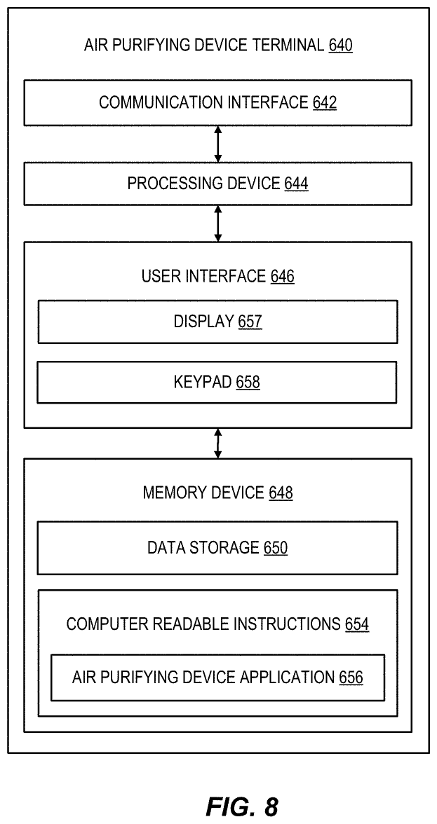

Referring now to FIG. 8, which schematically depicts an access terminal 112, in accordance with one embodiment of the invention, the access terminal 112 includes a communication interface 642 communicably coupled with a processing device 644, which is also communicably coupled with a memory device 648. The processing device 644 is configured to control the communication interface 642 such that the access terminal 112 communicates across the network 602 with one or more other systems, such as the user device 620 and the central server system 660. The processing device 644 is also configured to access the memory device 648 in order to read the computer readable instructions 654, which in some embodiments includes an air purifying device application 656. The air purifying device application 656, in some embodiments, allows for control of one or more operations of the primary air filtration unit 202, the particulate filtration unit 204, the secondary air filtration unit 206, and/or the humidifier unit 400 associated with the air purifying device 100. For example, the air purifying device application 656 may be used to configure and maintain the preset flow rate of air within the primary air filtration unit 202 and/or the secondary air filtration unit 206. The air purifying device application 656 may also allow for communication with the other systems and devices within the environment 100 such as the central server system 660. For example, the access terminal 112 may communicate over a network with one or more other systems to receive updates (e.g., firmware updates). The memory device 648 also includes a data storage or repository 650 or similar storage device for storing pieces of data that can be accessed by the processing device 644. The access terminal 112 further includes a user interface 646 for direct interaction with a user at the air purifying device 100 either alone or in combination with the user device 620. In one embodiment, the user interface 646 further includes a display 657 (e.g., a touchscreen display), a keypad 658, and/or the like.

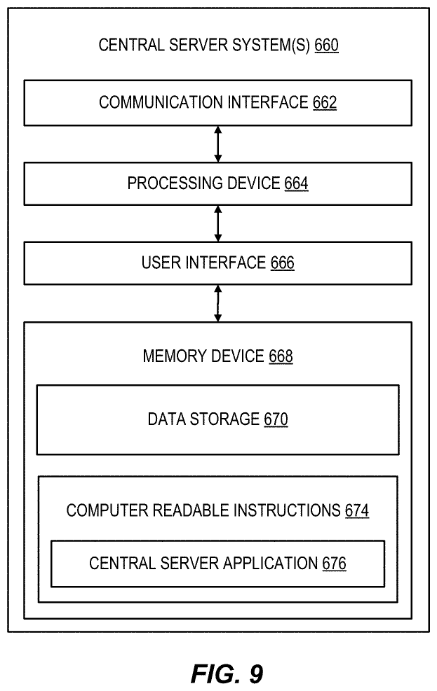

Referring now to FIG. 9, which schematically depicts a central server system, in accordance with one embodiment of the invention, the central server system 660 includes a processing device 664 operatively coupled to a communication interface 662 and a memory device 668. The processing device 664 is configured to control the communication interface 662 such that the central server system 660 communicates across the network 602 with a plurality of air purifying devices (such as the air purifying device 100), and/or one or more other systems. The processing device 664 is also configured to access the memory device 668 in order to read the computer readable instructions 674, which in some embodiments include a central server application 676. The central server application 676, in some embodiments, allows for authentication of a user requesting access to the air purifying device 100 and controls operation of the air purifying device 100. The memory device 668 also includes a data storage or repository 670 or similar storage device for storing pieces of data that can be accessed by the processing device 664.

The user application 634, the air purifying device application 656, and the central server application 676 are configured for instructing the processing devices on their respective systems to perform various steps of the methods discussed herein, and/or other steps and/or similar steps. In various embodiments, one or more of the various applications discussed are included in the computer readable instructions stored in a memory device of one or more systems or devices other than their respective systems and/or devices. For example, in some embodiments, the air purifying device application 656 may be stored and configured for being accessed by a processing device of the central server system 660 connected to the network 602. In various embodiments, the user application 634, the air purifying device application 656, and the central server application 676 are stored and executed by different systems/devices. In some embodiments, the discussed applications may be similar and may be configured to communicate with one another. In some embodiments, the various applications may be considered to be working together as a singular application despite being stored and executed on different systems.

In various embodiments, one of the systems discussed above, such as the central server system 660 or the access terminal 112, is more than one system and the various components of the system are not collocated, and in various embodiments, there are multiple components performing the functions indicated herein as a single device. For example, in one embodiment, multiple processing devices perform the functions of the processing device 664 of the central server system 660 described herein. In some embodiments, the one or more systems and/or applications described herein may communicate with one another bi-directionally, wherein commands, signals, messages, or the like may be transmitted and received between two or more of the systems and/or applications.

In various embodiments, the user device 620, the access terminal 112, and/or the central server system 660 may perform all or part of one or more method or process steps discussed herein and/or other method steps in association with the method steps discussed herein. Furthermore, some or all the systems/devices discussed herein, in association with other systems or without association with other systems, in association with steps being performed manually or without steps being performed manually, may perform one or more of the steps of one or more of the method discussed herein, or other methods, processes or steps discussed herein or not discussed herein.

As will be appreciated by one of ordinary skill in the art in view of this disclosure, the present invention may include and/or be embodied as an apparatus (including, for example, a system, machine, device, computer program product, and/or the like), as a method (including, for example, a business method, computer-implemented process, and/or the like), or as any combination of the foregoing. Accordingly, embodiments of the present invention may take the form of an entirely business method embodiment, an entirely software embodiment (including firmware, resident software, micro-code, stored procedures in a database, or the like), an entirely hardware embodiment, or an embodiment combining business method, software, and hardware aspects that may generally be referred to herein as a “system.” Furthermore, embodiments of the present invention may take the form of a computer program product that includes a computer-readable storage medium having one or more computer-executable program code portions stored therein. As used herein, a processor, which may include one or more processors, may be “configured to” perform a certain function in a variety of ways, including, for example, by having one or more general-purpose circuits perform the function by executing one or more computer-executable program code portions embodied in a computer-readable medium, and/or by having one or more application-specific circuits perform the function.

It will be understood that any suitable computer-readable medium may be utilized. The computer-readable medium may include, but is not limited to, a non-transitory computer-readable medium, such as a tangible electronic, magnetic, optical, electromagnetic, infrared, and/or semiconductor system, device, and/or other apparatus. For example, in some embodiments, the non-transitory computer-readable medium includes a tangible medium such as a portable computer diskette, a hard disk, a random access memory (RAM), a read-only memory (ROM), an erasable programmable read-only memory (EPROM or Flash memory), a compact disc read-only memory (CD-ROM), and/or some other tangible optical and/or magnetic storage device. In other embodiments of the present invention, however, the computer-readable medium may be transitory, such as, for example, a propagation signal including computer-executable program code portions embodied therein.

One or more computer-executable program code portions for carrying out operations of the present invention may include object-oriented, scripted, and/or unscripted programming languages, such as, for example, Java, Perl, Smalltalk, C++, SAS, SQL, Python, Objective C, JavaScript, and/or the like. In some embodiments, the one or more computer-executable program code portions for carrying out operations of embodiments of the present invention are written in conventional procedural programming languages, such as the “C” programming languages and/or similar programming languages. The computer program code may alternatively or additionally be written in one or more multi-paradigm programming languages, such as, for example, F #.

Some embodiments of the present invention are described herein with reference to flowchart illustrations and/or block diagrams of apparatus and/or methods. It will be understood that each block included in the flowchart illustrations and/or block diagrams, and/or combinations of blocks included in the flowchart illustrations and/or block diagrams, may be implemented by one or more computer-executable program code portions. These one or more computer-executable program code portions may be provided to a processor of a general purpose computer, special purpose computer, and/or some other programmable data processing apparatus in order to produce a particular machine, such that the one or more computer-executable program code portions, which execute via the processor of the computer and/or other programmable data processing apparatus, create mechanisms for implementing the steps and/or functions represented by the flowchart(s) and/or block diagram block(s).

The one or more computer-executable program code portions may be stored in a transitory and/or non-transitory computer-readable medium (e.g. a memory) that can direct, instruct, and/or cause a computer and/or other programmable data processing apparatus to function in a particular manner, such that the computer-executable program code portions stored in the computer-readable medium produce an article of manufacture including instruction mechanisms which implement the steps and/or functions specified in the flowchart(s) and/or block diagram block(s).

The one or more computer-executable program code portions may also be loaded onto a computer and/or other programmable data processing apparatus to cause a series of operational steps to be performed on the computer and/or other programmable apparatus. In some embodiments, this produces a computer-implemented process such that the one or more computer-executable program code portions which execute on the computer and/or other programmable apparatus provide operational steps to implement the steps specified in the flowchart(s) and/or the functions specified in the block diagram block(s). Alternatively, computer-implemented steps may be combined with, and/or replaced with, operator- and/or human-implemented steps in order to carry out an embodiment of the present invention.

Although many embodiments of the present invention have just been described above, the present invention may be embodied in many different forms and should not be construed as limited to the embodiments set forth herein; rather, these embodiments are provided so that this disclosure will satisfy applicable legal requirements. Also, it will be understood that, where possible, any of the advantages, features, functions, devices, and/or operational aspects of any of the embodiments of the present invention described and/or contemplated herein may be included in any of the other embodiments of the present invention described and/or contemplated herein, and/or vice versa. In addition, where possible, any terms expressed in the singular form herein are meant to also include the plural form and/or vice versa, unless explicitly stated otherwise. Accordingly, the terms “a” and/or “an” shall mean “one or more,” even though the phrase “one or more” is also used herein. Like numbers refer to like elements throughout.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the just described embodiments can be configured without departing from the scope and spirit of the invention. Therefore, it is to be understood that, within the scope of the appended claims, the invention may be practiced other than as specifically described herein.

What is claimed is:

1. An air purifying device comprising:
a housing defining an inlet aperture and an outlet aperture;
a primary filtration unit positioned within the housing, the primary filtration unit positioned proximate to the inlet aperture and configured to receive air through the inlet aperture for treatment;
a secondary filtration unit positioned within the housing, the secondary filtration unit positioned proximate to the outlet aperture and configured to exhaust air through the outlet aperture;
a particulate filtration unit positioned within the housing, the particulate filtration unit positioned between the primary filtration unit and the secondary filtration unit, the particulate filtration unit having an aperture that is capable of slidably receiving an ultra-low particle (ULPA) filter via an access opening;
wherein an airflow pathway is provided for flow of air from the primary filtration unit to the secondary filtration unit through the particulate filtration unit.

2. The air purifying device of claim 1, further comprising:
an inlet air distributor adapted to be detachably coupled to the housing proximate to the inlet aperture, wherein the inlet air distributor is configured to direct air into the inlet aperture for treatment.

3. The air purifying device of claim 2, further comprising:
an outlet air distributor adapted to be detachably coupled to the housing proximate to the outlet aperture, wherein the outlet air distributor is configured to direct air received from the outlet aperture.

4. The air purifying device of claim 1, further comprising:
an access terminal positioned on the housing, the access terminal being configured for controlling operation of the primary filtration unit, the secondary filtration unit, and the particulate filtration unit.

5. The air purifying device of claim 4, wherein the access terminal is configured to be in network communication with at least one of a user device and/or a central server, wherein the access terminal is configured to control the operation of the primary filtration unit, the secondary filtration unit, and the particulate filtration unit through the network communication with at least the user device and/or the central server.

6. The air purifying device of claim 1, wherein the particulate filtration unit comprises a plurality of stabilizing elements arranged on an interior wall of the aperture, the plurality of stabilizing elements is configured to slidably receive the ultra-low particle air (ULPA) filter such that when the ultra-low particle air (ULPA) filter is inserted into the particulate filtration unit through the access opening, the plurality of stabilizing elements form a secure seal with at least a top edge and a bottom edge of the ultra-low particle air (ULPA) filter.

7. The air purifying device of claim 1, wherein the primary filtration unit further comprises:
a high efficiency particulate air (HEPA) filter, the high efficiency particulate air (HEPA) filter positioned proximate to the inlet aperture and configured to pre-filter airborne particulate matter from the air received from the inlet aperture;
an inlet airflow sensor, the airflow sensor positioned proximate to the high efficiency particulate air (HEPA) filter and configured to receive air filtered by the high efficiency particulate air (HEPA) filter;
an airflow regulation device; and
a plurality of ultraviolet light sources, the plurality of ultraviolet light sources is adapted to be positioned longitudinally within the primary filtration unit,
wherein the airflow regulation device is configured to regulate and direct the flow of air filtered by the high efficiency particulate air (HEPA) filter towards the plurality of ultraviolet light sources.

8. The air purifying device of claim 7, wherein the high efficiency particulate air (HEPA) filter is configured to pre-filter airborne particulate matter of a size equal to or greater than 0.3 micron from the air received from the inlet aperture.

9. The air purifying device of claim 8, wherein the inlet airflow sensor is configured to measure a flow rate of air filtered by the high efficiency particulate air (HEPA) filter.

10. The air purifying device of claim 9, wherein the airflow regulation device is configured to regulate and direct the flow of air filtered by the high efficiency particulate air (HEPA) filter towards the plurality of ultraviolet light sources based on at least the flow rate of air measured by the inlet airflow sensor.

11. The air purifying device of claim 10, wherein the plurality of ultraviolet light sources is configured to generate short wave ultraviolet radiation (UV-C) to treat the air filtered by the high efficiency particulate air (HEPA) filter and displaced by the airflow regulation device.

12. The air purifying device of claim 11, wherein the ultra-low particle air (ULPA) filter is configured to filter airborne particulate matter from the air passed through the plurality of ultraviolet light sources in the primary filtration unit, wherein the air is moved from the primary filtration unit to the particulate filtration unit via the airflow pathway.

13. The air purifying device of claim 12, wherein the ultra-low particle air (ULPA) filter is configured to filter airborne particulate matter of a size equal to or greater than 0.12 micron from the air received from the primary filtration unit.

14. The air purifying device of claim 1, wherein the secondary filtration unit further comprises:
a plurality of ultraviolet light sources, the plurality of ultraviolet light sources is adapted to be positioned longitudinally within the secondary filtration unit;
an airflow regulation device; and
an outlet airflow sensor, the outlet airflow sensor positioned proximate to the outlet aperture and configured to receive air from the airflow regulation device,
wherein the airflow regulation device is configured to regulate and direct the flow of air from the plurality of ultraviolet light sources towards the outlet airflow sensor and the outlet aperture.

15. The air purifying device of claim 14, wherein the outlet airflow sensor is configured to measure a flow rate of air received from the airflow regulation device.

16. The air purifying device of claim 15, wherein the airflow regulation device is configured to regulate and direct the flow of air from the plurality of ultraviolet light sources towards outlet aperture based on at least the flow rate of air measured by the outlet airflow sensor.

17. The air purifying device of claim 16, wherein the plurality of ultraviolet light sources is configured to generate short wave ultraviolet radiation (UV-C) to treat the air received via the airflow pathway from the particulate filtration unit.

18. The air purifying device of claim 1, further comprising:
a humidifier unit positioned within the housing, the humidifier unit configured to infuse moisture into the air prior to exhausting the air through the outlet aperture, the humidifier unit further comprising:
a plurality of water filters, the plurality of water filters configured to remove waterborne contaminate matter to produce filtered water;
a water basin, the water basin configured to receive filtered water from the plurality of water filters;
a float valve, the float value configured to measure a water level in the water basin; and
a wick cartridge, the wick cartridge configured to absorb the filtered water from the water basin, and wherein the wick cartridge is configured to infuse moisture into the air when the air is passed through the wick cartridge.

19. The air purifying device of claim 1, wherein the housing is adapted to be mounted in line with or intermediate of a plurality of framing members of a wall in a concealed configuration.

20. The air purifying device of claim 19, wherein the housing is adapted to be mounted on the surface of the wall in an exposed configuration.
